(19) United States
(12) Patent Application Publication
(10) Pub. No.: US 2014/0249153 A1
(43) Pub. Date: Sep. 4, 2014

(54) FIXED DOSE COMBINATION OF BIMATOPROST AND BRIMONIDINE

(71) Applicants: Richard S. Graham, Irvine, CA (US); Chetan P. Pujara, Irvine, CA (US); Sesha Neervannan, Irvine, CA (US); Anuradha V. Gore, Aliso Viejo, CA (US); Kevin S. Warner, Anaheim, CA (US)

(72) Inventors: Richard S. Graham, Irvine, CA (US); Chetan P. Pujara, Irvine, CA (US); Sesha Neervannan, Irvine, CA (US); Anuradha V. Gore, Aliso Viejo, CA (US); Kevin S. Warner, Anaheim, CA (US)

(73) Assignee: Allergan, Inc., Irvine, CA (US)

(21) Appl. No.: 14/235,746

(22) PCT Filed: Jul. 20, 2012
(86) PCT No.: PCT/US12/47586

§ 371 Date: Mar. 4, 2014

Related U.S. Application Data
(60) Provisional application No. 61/509,666, filed on Jul. 20, 2011.

(51) Int. Cl. A61K 31/5575; A61K 31/498

(52) U.S. Cl. CPC A61K 31/5575; A61K 31/498
USPC 514/249

(57) ABSTRACT

The present invention is directed to compositions comprising combinations of brimonidine and bimatoprost useful for lowering intraocular pressure in a patient and for the treatment of glaucoma.

### RELATED APPLICATION

This application claims the benefit of U.S. Provisional Application Ser. No. 61/509,666, filed Jul. 20, 2011, the disclosure of which is hereby incorporated in its entirety herein by reference.

### FIELD OF THE INVENTION

The present application is directed to composition comprising combinations of brimonidine and bimatoprost useful for lowering intraocular pressure in a patient and the treatment of glaucoma.

### BACKGROUND OF THE INVENTION

Topically applied formulations (defined as formulations applied to the cornea, conjunctiva, etc.) are frequently used in ophthalmology to treat acute and chronic conditions because they are considered to be safer relative to systemically delivered formulations.

While topically applied formulations may not produce a high systemic exposure of the active pharmaceutical ingredient, there is still the potential for adverse events (e.g., conjunctival hyperemia) due to topical exposure. Improving the side effect profile while still maintaining and possibly improving efficacy can be accomplished via the following: 1) reduce the concentration of the API to the lowest effective dose; 2) include a second API with a mechanism of action known to minimize the adverse event of the first API; 3) Include a second API which will provide a synergistic effect thereby improving the overall efficacy; and 4) improve patient compliance by reducing the number of different medications that need to be delivered. Specifically, this invention discloses the fixed dose combination of bimatoprost and brimonidine in an appropriate formulation vehicle.

### SUMMARY OF THE INVENTION:

Formulation development approaches to meet the above criteria for bimatoprost/brimonidine are described as follows:
1. Optimize the pH of the formulation to maximize the unionized fraction of brimonidine in aqueous formulations;
2. Incorporation of viscosity agents in the formulation to increase solution viscosity; bioavailability; and,
4. Non-aqueous based formulations.

The present invention is intended for use in patients who require more than one intraocular pressure lowering agent and/or to improve patient compliance for patients undergoing concurrent bimatoprost and brimoindine monotherapy.

“About” is defined as variations in amounts in either active compounds or excipients that would be considered bioequivalent by a regulatory agency such as the FDA or the EMEA.

“Effective amount” An “effective amount” of a compound is an amount sufficient to contribute to the treatment, prevention, or reduction of a symptom or symptoms of a disease. Where recited in reference to a disease treatment, an “effective amount” may also be referred to as a “therapeutically effective amount.” A “reduction” of a symptom or symptoms (and grammatical equivalents of this phrase) means decreasing of the severity or frequency of the symptom(s), or elimination of the symptom(s).

A “pharmaceutically acceptable carrier” or “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Na—CMC” means sodium carboxymethyl cellulose and can be either low density, medium density or high density CMC and mixtures thereof.

The term “pharmaceutically acceptable salts” is meant to include salts of the active compounds which are prepared with relatively nontoxic acids or bases, depending on the particular substituents found on the compounds described herein.

“Soluplus®” refers to the solubilizer sold by BASF known as polyvinyl capralactam-polyvinyl acetate-polyethylene glycol graft copolymer (PCA-PVA-PEG).

The term “topical” in the context of methods described herein relates in the customary sense to the administration of a compound or pharmaceutical composition which is incorporated into a suitable pharmaceutical carrier and administered at a topical treatment site of a subject. Accordingly, the term “topical pharmaceutical composition” includes those with a topical treatment site, e.g., the eye or the skin. The term “topical ocular pharmaceutical composition” refers to a pharmaceutical composition suitable for administering directly to the eye.

The terms “treating” or “treatment” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being.

Some embodiments of the invention include:
1) A topical composition for use in lowering IOP in a patient comprising an effective amount of bimatoprost and brimonidine and a pharmaceutically acceptable carrier.
2) The composition of embodiment 1 wherein the bimatoprost is present in the concentration range of 0.001-0.03% w/w and the brimonidine is brimonidine tartrate and is present in the concentration range of 0.005-0.2% w/w.
3) The composition of embodiments 1-2 wherein the bimatoprost is present in the concentration of about 0.01% w/w and the brimonidine is present in the amount of about 0.1% w/w.
4) The composition of embodiments 1-3 wherein the composition further comprises excipients selected from the group of excipients listed in Table 1, Table 2 and Table 3.
5) The compositions of embodiment 4 wherein the excipients are present in concentrations at about the concentrations listed in Tables I, II and III.
6) A method of lowering intraocular pressure or treating glaucoma in a human patient comprising administering the composition of embodiment 3.
7) A method of lowering intraocular pressure or treating glaucoma in a human patient comprising administering a composition of embodiments 1-6.
8) A method of lowering intraocular pressure or treating glaucoma in a human patient comprising administering one of the compositions listed in Table 1.
9) A method of lowering intraocular pressure or treating glaucoma in a human patient comprising administering one of the compositions listed in Table 2 or Table 3.
10) The method of embodiments 1-9 wherein the composition is administered once a day. day or more.
12) A topical composition for ophthalmic application for use in lowering intraocular pressure in a patient comprising about 0.01% w/w bimatoprost and about 0.1% w/w brimonidine.
13) The composition of embodiment 12 wherein the bimatoprost is present in the concentration of 0.01% w/w and brimonidine is brimonidine tartrate and is present in the amount of 0.1% w/w in an aqueous vehicle.
14) The composition of embodiment 12 further comprising a solubulizer selected from the group consisting of Na—CMC and Soluplus®.
15) The composition of embodiment 12 further comprising buffers selected from the group consisting of sodium phosphate dibasic heptahydrate, citric acid monohydrate, sodium borate decahydrate, sodium hydroxide and hydrochloric acid.
16) The compositions of embodiment 12 further comprising tonicity agents wherein the tonicity agents are selected from the group consisting of NaCl and glycerin.
17) The composition of embodiment 12 further comprising preservatives wherein the preservatives are selected from the group consisting of benzalkoniun chloride and Purite.
18) The topical composition of embodiment 12 wherein the composition is a solution and is administered at least once a day.

19) The topical composition of embodiment 12 wherein the composition is administered twice a day.
20) The topical composition of embodiments 12-17 wherein the composition is useful in treating glaucoma wherein the glaucoma is selected from the group consisting of open-angle glaucoma, closed-angle glaucoma, angle-closure glaucoma, normal-tension glaucoma, and congenital glaucoma.
21) The composition of embodiment 12 wherein the composition of embodiment 12 lowers intraocular pressure more than either bimatoprost or brimonidine administered alone and with fewer side-effects.
22) A method of treating elevated intraocular pressure the method comprising administering a composition to a patient suffering elevated intraocular pressure wherein the composition comprises about 0.01% w/w bimatoprost and about 0.1% w/w brimonidine tartrate. bimatoprost and 0.1% w/w brimonidine.
24) The method of embodiment 22 wherein the composition further comprises a solubulizer selected from the group consisting of Na—CMC and Soluplus® and a buffer selected from the group consisting of sodium phosphate dibasic heptahydrate, citric acid monohydrate, sodium borate decahydrate, sodium hydroxide and hydrochloric acid.
25) The method of embodiments 22-24 wherein the composition of claim 11 lowers intraocular pressure more effectively than either brimonidine monotherapy (0.2%, 0.15%, or 0.1%) or bimatoprost monotherapy (0.03% or 0.01%).
26) The method of embodiments 22-24 wherein the composition of claim 11 lowers intraocular pressure more effectively than either brimonidine monotherapy or bimatoprost monotherapy and with fewer overall ocular side effects than either brimonidine and bimatoprost monotherapy.
27) The method of embodiments 22-24 wherein the method is effective in treating glaucoma.
28) The method of embodiments 22-24 wherein the method is effective in lower ocular hypertension.
29) The method of embodiments 22-24 wherein the composition further comprises a preservative.
30) The method of embodiments 22-24 wherein the composition is administered at least once a day.
31) The method of embodiment 22 wherein the composition is applied topically to the eye.

TABLE 1
Formulation Examples:
Example #

| | 1 | 2 | 3 | 4 | 5 | 6 | 7 | 8 | 9 | 10 | 11 |
|---|---|---|---|---|---|---|---|---|---|---|---|
| Active | % (w/w) | | | | | | | | | | |
| Ingredients | | | | | | | | | | | |
| Bimatoprost | 0.01 | | | | | | | | | | |
| Brimonidine | 0.1 | | | | | | | | | | |
| Excipients | % (w/w) | | | | | | | | | | |
| Hydroxyethyl | | | | | | | | | | 0.3-1 | |
| cellulose | | | | | | | | | | | |
| Sodium | 0.268 | 0.268 | 1.5 | | 0.268 | 0.268 | 0.268 | 0.268 | 0.268 | 0.268 | 0.268 |
| phosphate | | | | | | | | | | | |
| dibasic | | | | | | | | | | | |
| Citric acid | 0.014 | 0.014 | 0.025 | | 0.014 | 0.014 | 0.014 | 0.014 | 0.014 | 0.014 | 0.014 |
| Glycerin | QS | | | | QS | | | QS | QS | QS | QS |
| | Osm | | | | Osm | | | Osm | Osm | Osm | Osm |
| | 270-320 | | | | 270-320 | | | 270-320 | 270-320 | 270-320 | 270-320 |
| NaCl | | QS | QS | QS | | QS | QS | | | | |
| | | Osm | Osm | Osm | | Osm | Osm | | | | |
| | | 270-320 | 270-320 | 270-320 | | 270-320 | 270-320 | | | | |
| EDTA | | | | | 0.01 | 0.01 | | | | | 0.01 |
| Select one or | | | | | 0.001-1 | 0.001-1 | | | | | 0.5 |
| more from the | | | | | | | | | | | |
| following: | | | | | | | | | | | |
| Solutol; | | | | | | | | | | | |
| Tween 20, 40, | | | | | | | | | | | |
| 60, or 80; | | | | | | | | | | | |
| Poloxamer, | | | | | | | | | | | |
| POE 40 | | | | | | | | | | | |
| Stearate; | | | | | | | | | | | |
| Castor Oil | | | | | | | | | | | 0.1 |
| Sodium borate | | | | 0.045 | | | | | | | |
| Boric acid | | | | 0.6 | | | | | | | |
| pH | 7.0-7.8 | 7.0-7.8 | 7.0-7.8 | 7.0-7.8 | 7.0-7.8 | 7.0-7.8 | 7.0-7.8 | 7.0-7.8 | 7.0-7.8 | 7.0-7.8 | 7.0-7.8 |
| BAK | 0.005 | 0.005 | 0.005 | | 0.005 | 0.005 | | 0.003 | 0.02 | 0.005 | 0.005 |
| Purite | | | | 0.01 | | | 0.01 | 0.005 | | | |
| Water | QS 100% | QS 100% | QS 100% | | QS 100% | QS 100% | QS 100% | QS 100% | QS 100% | QS 100% | QS 100% |

Example #

| | | | |
|---|---|---|---|
| 12 | 13 | 14 | 15 |

| | |
|---|---|
| Active Ingredients | % (w/w) |
| Bimatoprost | 0.01 |
| Brimonidine | 0.1 |
| Excipients | % (w/w) |

| | | | | |
|---|---|---|---|---|
| White petrolatum | QS 100 | | | |
| Mineral Oil | 30-40 | | | 5-10 |
| Lanolin | 5-15 | 5-15 | | |
| Beeswax | | 10-20 | | 10-30 |
| ST-Wax 30 | | 8-12 | | 5-10 |
| Cetyl Alcohol | | 5-10 | | |
| Castor Oil | | QS 100 | | QS 100 |
| Jojoba Seed Oil | | | | 5-10 |
| Silky Wax 10 | | | 5-15 | |
| Cyclomethicone 5-NF | | | QS 100 | |
| Caprylic/capric | | | 5-15 | |
| triglyceride | | | | |
| Isopropyl myristate | | | 5-15 | |

TABLE 2
Further Formulation Examples:

| | Lipid Nanoparticle | Silicone with dispersed lipid nanoparticle |
|---|---|---|
| Active Ingredients | w/w % | |
| Bimatoprost | 0.01 | |
| Brimonidine | 0.1 | |
| Excipients | % (w/w) | |
| Silicone fluid | | QS 100% |
| Dimethiconol blend 20 | | 0-20 |
| Crodamol MM | 10-40 | |
| Water | QS 100% | |
| Castor Oil | | 0-5 |
| Solutol HS | 0.01-5 | |
| Glycerin | QS to 270-320 mOsm | |
| BAK | 0.01 | |

The formulations of the present invention can be topically administered once, twice or three times a day in order to lower intraocular pressure in a patient.

The present formulations may be preserved or preservative free. Although the concentrations of actives in Tables 1 and 2 are preferred, bimatoprost may be present in w/w. Concentrations of actives and excipients may be present in about the concentrations listed herein, wherein “about” refers to variations of the concentrations considered to be bioequivalent by the FDA or EMEA in making similar or generic compositions.

Brimonidine includes pharmaceutically acceptable salts of brimonidone such as brimonidine tartrate. Brimonidine tartrate is an alpha adrenergic agonist represented by the following formula:

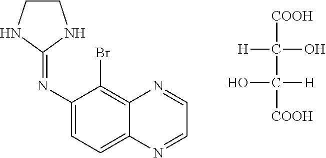 [SMILES: Brc1c(N=C2NCCN2)ccc2nccnc12 ; O=C(O)C(O)C(O)C(=O)O]

The chemical name for brimonidine is 5-Bromo-6-(2-imizazolidinylideeneamno)quinoxaline L-tartrate.

Bimatoprost is represented by the following chemical structure:

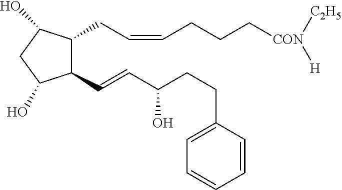

Bimatoprost's chemical name is (Z)-7-R1R,2R,3R,5S)-3,5-Dihydroxy-2-[(1E,3S)-3-hydroxy-5-phenyl-1-pentenyl]cyclopentyl]-5-N-ethylheptenamide, and its molecular weight is 415.58. Its molecular and its formula is C₂₅H₃₇NO₄.

TABLE 3
Brimonidine/Bimatoprost Fixed Dose Combination Formulations for Stability Evaluation

| | | Formulation# | | | | | | | |
|---|---|---|---|---|---|---|---|---|---|
| | | 1 | 2 | 3 | 4 | 5 | 6 | 7 | 8 |
| Active | Bimatoprost | 0.01 | 0.01 | 0.01 | 0.01 | 0.01 | 0.01 | 0.01 | 0.01 |
| Ingredients | Brimonidine | 0.1 | 0.1 | 0.1 | 0.1 | 0.1 | 0.1 | 0.1 | 0.1 |
| (% w/w) | | | | | | | | | |
| Solubilizers | Na-CMC (Low | | | 0.5 | 0.5 | | | | |
| (% w/w) | viscosity | | | | | | | | |
| | (190342-LF) | | | | | | | | |
| | 7LFPH) | | | | | | | | |
| | Soluplus ® | 1 | 1 | | | | | | 1 |
| Buffers | Sodium | 0.268 | | 0.268 | | 0.268 | 0.268 | 0.268 | 0.268 |
| (% w/w) | phosphate | | | | | | | | |
| | dibasic | | | | | | | | |
| | heptahydrate | | | | | | | | |
| | Citric acid | 0.014 | | 0.014 | | 0.014 | 0.014 | 0.014 | 0.014 |
| | monohydrate | | | | | | | | |
| | Sodium borate | | 0.095 | | 0.095 | | | | |
| | decahydrate | | | | | | | | |
| | Boric acid | | 0.229 | | 0.229 | | | | |
| | Target pH | 7.7 (7.4-8.0) | 7.7 (7.4-8.0) | 7.7 (7.4-8.0) | 7.7 (7.4-8.0) | 7.0 (6.7-7.3) | 7.0 (6.7-7.3) | 7.0 (6.7-7.3) | 7.7 (7.4-8.0) |
| | 1N Sodium | QS to pH | QS to pH | QS to pH | QS to pH | QS to pH | QS to pH | QS to pH | QS to pH |
| | Hydroxide/ | | | | | | | | |
| | 1N Hydrochloric | | | | | | | | |
| | Acid | | | | | | | | |
| Tonicity | NaCl | | | 0.8 | 0.8 | 0.8 | 0.8 | 0.8 | 0.8 |
| Modifiers | Glycerin | 2.3 | 2.3 | | | | | | |
| (% w/w) | | | | | | | | | |
| Preservatives | BAK | 0.005 | | | | 0.02 | 0.01 | 0.003 | 0.01 |
| (% w/w) | Purite | | 0.01 | | | | | 0.01 | |
| Vehicle | Purified Water | Q.S. to 100 | Q.S. to 100 | Q.S. to 100 | Q.S. to 100 | Q.S. to 100 | Q.S. to 100 | Q.S. to 100 | Q.S. to 100 |
| (% w/w) | | | | | | | | | |

Formulation stability for the formulation shown in Table 3 was as follows:

### Formulation Stability

TABLE 4
Potency of Brimonidine in B2 Formulations (expressed as percent of label strength)
over 3 months:

| | | Time | 1 Month | | 3 Month | |
|---|---|---|---|---|---|---|
| Stability | | Zero | 25 C./40% RH | 40 C./25% RH | 25 C./40% RH | 40 C./25% RH |
| Soluplus ® | Formulation 1 | 100.3 | 102.1 | 101.7 | 99.7 | 100.2 |
| containing | Formulation 2 | 99.4 | 101.4 | 102.2 | N/A | N/A |
| formulation, | Formulation 8 | 100.9 | 101.9 | 104.1 | 101.8 | 102.6 |
| pH 7.7 | | | | | | |
| Na CMC | Formulation 3 | 99.7 | 101.7 | 103.3 | 101.4 | 101.3 |
| containing | Formulation 4 | 99.5 | 102.1 | 102.6 | 100.4 | 102.1 |
| formulation, | | | | | | |
| pH 7.7 | | | | | | |
| pH 7.0 | Formulation 5 | 101.6 | 102.5 | 104.1 | 101.5 | 103.5 |
| formulation | Formulation 6 | 102.4 | N/A | N/A | 102.1 | 101.6 |
| | Formulation 7 | 101.6 | 101.9 | 104.1 | N/A | N/A |

TABLE 5
Potency of Bimatoprost in B2 Formulations (expressed as percent of label strength)
over 3 months

| | | Time | 1 Month | | 3 Month | |
|---|---|---|---|---|---|---|
| Stability | | Zero | 25 C./40% RH | 40 C./25% RH | 25 C./40% RH | 40 C./25% RH |
| Soluplus ® | Formulation 1 | 101.6 | 99.8 | 100.5 | 99.0 | 100.4 |
| containing | Formulation 2 | 88.6 | 82.1 | 81.8 | N/A | N/A |
| formulation, | Formulation 8 | 100.1 | 97.1 | 98.6 | 96.6 | 97.3 |
| pH 7.7 | | | | | | |
| Na CMC | Formulation 3 | 100.5 | 102.4 | 101.4 | 98.6 | 100.6 |
| containing | Formulation 4 | 101.5 | 100.7 | 102.0 | 99.1 | 99.7 |
| formulation, | | | | | | |
| pH 7.7 | | | | | | |
| pH 7.0 | Formulation 5 | 100.9 | 99.2 | 96.9 | 99.6 | 100.8 |
| formulation | Formulation 6 | 100.0 | N/A | N/A | 97.9 | 99.8 |
| | Formulation 7 | 98.6 | 96.1 | 98.6 | N/A | N/A |

Note:
Formulation 2 and 7 that contain purite were discontinued after 1 month pull due to drug degradation.

TABLE 6
Brimonidine/Bimatoprost Fixed Dose Combination Formulations for Rabbit PK Assessments

| | | Soluplus ® Containing Formulation | | | | | | | Control | |
|---|---|---|---|---|---|---|---|---|---|---|
| | | (pH 7.7) | | | | NaCMC | pH 7.0 | | | Lumi- |
| | | | | | SP 100 | (pH 7.7) | Formulation | | Alphagan | gan |
| Formulation | | SP 0 | SP 50 | SP 100 | NaCl | NaCMC 0 | LP 50 | LP 100 | 0.2% | 0.03% |
| Active | Bimatoprost | 0.01 | 0.01 | 0.01 | 0.01 | 0.01 | 0.01 | 0.01 | | 0.03 |
| Ingredients | Brimonidine | 0.1 | 0.1 | 0.1 | 0.1 | 0.1 | 0.1 | 0.1 | 0.2 | |
| (% w/w) | Tartrate | | | | | | | | | |
| Solubilizers | Na-CMC | | | | | 0.5 | | | | |
| (% w/w) | Soluplus ® | 1 | 1 | 1 | 1 | | | | | |
| Buffers | Sodium | 0.268 | 0.268 | 0.268 | 0.268 | 0.268 | 0.268 | 0.268 | | 0.268 |
| (% w/w) | phosphate | | | | | | | | | |
| | dibasic | | | | | | | | | |
| | heptahydrate | | | | | | | | | |
| | Citric acid | 0.014 | 0.014 | 0.014 | 0.014 | 0.014 | 0.014 | 0.014 | 0.050 | 0.014 |
| | monohydrate | | | | | | | | | |
| | Sodium | | | | | | | | 0.45 | |
| | Citrate | | | | | | | | | |
| | Dihydrate | | | | | | | | | |
| | Target pH | 7.7 | 7.7 | 7.7 | 7.7 | 7.7 | 7.0 | 7.0 | 6.3 | 7.3 |
| Tonicity | NaCl | | | | 0.8 | 0.80 | 0.80 | 0.8 | 0.70 | 0.83 |
| Modifiers | Glycerin | 2.3 | 2.3 | 2.3 | | | | | | |
| (% w/w) | | | | | | | | | | |
| Preservatives | BAK | | 50 | 100 | 100 | | 50 | 100 | 50 | 50 |
| (ppm) | | | | | | | | | | |
| Vehicle | Purified | Q.S. to 100 | Q.S. to 100 | Q.S. to 100 | Q.S. to 100 | Q.S. to 100 | Q.S. to 100 | Q.S. to 100 | | |
| (% w/w) | Water | | | | | | | | | |

TABLE 7
Brimonidine and Bimatoprost in Aqueous Humor

| | | | Brimonidine | | | Bimatoprost | | |
|---|---|---|---|---|---|---|---|---|
| | | | Tmax | Cmax | AUClast | Tmax | Cmax | AUClast |
| Matrix | Treatment | | (hr) | (ng/mL) | (ng · hr/mL) | (hr) | (ng/mL) | (ng · hr/mL) |
| Aqueous | Control | Alphagan | 1 | 472 | 927 | | | |
| Humor | | 0.2% BID | | | | | | |
| | pH 7.0 | LP 50 BID | 0.5 | 295 | 494 | 1 | 9.46 | 35.1 |
| | Formulation | LP 100 | 0.5 | 296 | 628 | 2 | 23.4 | 63.6 |
| | | BID | | | | | | |
| | Control | Lumigan | | | | 1 | 39.9 | 140 |
| | | 0.03% QD | | | | | | |
| | NaCMC pH | NaCMC 0 | 0.5 | 463 | 758 | 1 | 9.44 | 34.3 |
| | 7.7 | BID | | | | | | |
| | Soluplus ® | SP 0 BID | 0.5 | 349 | 611 | 2 | 5.16 | 16.7 |
| | Containing | SP 50 BID | 0.5 | 304 | 662 | 2 | 7.95 | 24.1 |
| | Formulation, | SP 100 | 0.5 | 445 | 677 | 2 | 6.35 | 23.9 |
| | pH 7.7 | BID | | | | | | |
| | | SP 100 | 0.5 | 428 | 688 | 1 | 7.80 | 26.0 |
| | | NaCl BID | | | | | | |

LLOQ (AQH) = 0.1 ng/mL

### EXAMPLE

### Example 1

A 58 year old Caucasian male with elevated intraocular pressure (“IOP”) is unresponsive to both brimonidine (0.15% w/v and 0.01% w/v) and bimatoprost monotherapy (both 0.03% w/v and 0.01% w/v) and unable to adequately control his elevated IOP. The 58 year old male administers Formulation 6, in Table 3 twice a day, once in the morning and once in the evening. Administration is 12 hours apart and every day. Within three days of use, the patient's IOP falls to clinically acceptable levels and remains at clinically acceptable levels as long as the patient applies Formula 6 twice a day.

### Example 2

a 71 year old African American female with ocular hypertension is unresponsive to both brimonidine and bimatoprost monotherapy and unable to control her IOP through the use of conventional glaucoma medications. The 71 year old patient administers Formulation 8, in Table 3, once each day. Within seven days of use, the patient's IOP falls to clinically acceptable levels and remains at clinically acceptable levels for over 120 days of daily administration of Formulation 8.

### Example 3

A 68 year old Caucasian male with elevated intraocular pressure, open-angle glaucoma and with sensitivity to ophthalmic preservatives is administered Formulation 3 in Table 3 on a once daily basis. After several days of use, the patients intraocular pressure drops to therapeutically acceptable levels and stays at therapeutically acceptable levels so long as daily administration of Formulation 3 is continued. After 6 months of daily use of Formulation 3, there is no further worsening of the patient's glaucoma and no further detectable damage to the optic nerve.

### Example 4

A 73 Hispanic female suffering ocular hypertension ranging from 17-20 mm Hg is unresponsive to commercially available brimonidine and bimatoprost monotherapy. The patient is administered Formulation 5 of Table 3 once a day and after two days the patient's intraocular pressure lowers to acceptable levels. The patient continues administering Formulation 5 every day and intraocular pressure levels remained at therapeutically acceptable levels.

1. A topical composition for ophthalmic application for use in lowering intraocular pressure in a patient comprising about 0.01% w/w bimatoprost and about 0.1% w/w brimonidine.

2. The composition of claim 1 wherein the bimatoprost is present in the concentration of 0.01% w/w and brimonidine is brimonidine tartrate and is present in the amount of 0.1% w/w in an aqueous vehicle.

3. The composition of claim 2 further comprising a solubilizer selected from the group consisting of Na—CMC and Soluplus®.

4. The composition of claim 2 further comprising buffers selected from the group consisting of sodium phosphate dibasic heptahydrate, citric acid monohydrate, sodium borate decahydrate, sodium hydroxide and hydrochloric acid.

5. The compositions of claim 2 further comprising tonicity agents wherein the tonicity agents are selected from the group consisting of NaCl and glycerin.

6. The composition of claim 2 further comprising preservatives wherein the preservatives are selected from the group consisting of benzalkoniun chloride and Purite.

7. The topical composition of claim 2 wherein the composition is a solution and is administered at least once a day.

8. The topical composition of claim 2 wherein the composition is administered twice a day.

9. The topical composition of claim 2 wherein the composition is useful in treating glaucoma wherein the glaucoma is selected from the group consisting of open-angle glaucoma, closed-angle glaucoma, angle-closure glaucoma, normal-tension glaucoma, and congenital glaucoma.

10. The composition of claim 2 wherein the composition of claim 2 lowers intraocular pressure more than either bimatoprost or brimonidine administered alone and with fewer side-effects.

11. A method of treating elevated intraocular pressure the method comprising administering a composition to a patient suffering elevated intraocular pressure wherein the composition comprises about 0.01% w/w bimatoprost and about 0.1% w/w brimonidine tartrate.

12. The method of claim 11 wherein the composition comprises 0.01% w/w bimatoprost and 0.1% w/w brimonidine.

13. The method of claim 11 wherein the composition further comprises a solubulizer selected from the group consisting of Na—CMC and Soluplus® and a buffer selected from the group consisting of sodium phosphate dibasic heptahydrate, citric acid monohydrate, sodium borate decahydrate, sodium hydroxide and hydrochloric acid.

14. The method of claim 11 wherein the composition of claim 11 lowers intraocular pressure more effectively than either brimonidine monotherapy or bimatoprost monotherapy.

15. The method of claim 14 wherein the composition of claim 11 lowers intraocular pressure more effectively than either brimonidine monotherapy or bimatoprost monotherapy and with fewer overall ocular side effects than either brimonidine and bimatoprost monotherapy.

16. The method of claim 11 wherein the method is effective in treating glaucoma.

17. The method of claim 11 wherein the method is effective in lower ocular hypertension.

18. The method of claim 11 wherein the composition further comprises a preservative.

19. The method of claim 11 wherein the composition is administered at least once a day.

20. The method of claim 11 wherein the composition is applied topically to the eye.
